(19) United States
(12) Patent Application Publication
(10) Pub. No.: US 2025/0229033 A1
(43) Pub. Date: Jul. 17, 2025

(54) INJECTION ENDPOINT MONITORING SYSTEM AND METHOD

(71) Applicant: BIOCORP PRODUCTION S.A.S., Issoire (FR)

(72) Inventors: Alain MARCOZ, Issoire (FR); Lionel ALDON, Issoire (FR); Gael SENEE, Issoire (FR)

(21) Appl. No.: 18/848,550

(22) PCT Filed: Apr. 28, 2022
(86) PCT No.: PCT/IB2022/000265

§ 371 Date: Sep. 19, 2024

(51) Int. Cl. A61M 5/315

(52) U.S. Cl. CPC A61M 5/31568; A61M 5/31585; A61M 2205/3317; A61M 2205/50

(57) ABSTRACT

An injection endpoint monitoring system is configured for integration into an injection monitoring module mounted on a proximal end of a pen injection system comprising a proximal activation button. The monitoring system comprises a processor, a magnetometer, and an accelerometer, electrically connected to the processor, which is configured to receive data and/or information from either the magnetometer and/or the accelerometer. A magnetic field generator is configured to provide a magnetic field detected by the magnetometer, and is rotatable about a first axis, and located on, and translatable along, the first axis. The processor is configured to process data and/or information received synchronously from both the magnetometer and the accelerometer to determine whether a dose setting operation has been effected, whether a translational movement of the accelerometer along the first axis of the system has occurred, and to determine an endpoint of an injection operation of the pen injection system.

The present invention relates generally to systems and methods for the determination an injection endpoint.

Generally, being able to determine when an injection endpoint has been reached is highly desirable, for a number, of reasons, not least that of safety with regard to a user of an injection system, or a patient to whom an injection is administered. Various methods and systems are already known generally in the art, for example, in infusion systems. One area of more particular interest is being able to determine, or monitor an injection endpoint event in a handheld injection device, such as a pen injection system.

As used herein, the terms “pen injection system” and “injection pen system” are used interchangeably to designate a generally handheld pen-shaped injection system, such systems being readily well known per se and commercially available for use in the treatment or management of many various medical indications. These systems are also often generally designed for self-injection of a drug by the user in need of treatment for, or management of, the given medical indication. This is for example the case with insulin, supplied in various forms for use in the treatment of diabetes, for example the pen injection systems commercialized under a variety of brand names, such as FlexPen® commercialized by Novo Nordisk, Kwikpen® commercialized Eli Lilly, or Lantus Solostar® commercialized by Sanofi, being but three such pen injections among the most well known. Other drugs are also used with this category of medical devices, and are required, for example, to address other medical conditions such as hormone-deficiency or hormone over-production related conditions, and potentially life-threatening situations, enabling immediate emergency injection of a required drug, such as anaphylactic shock treatments, anti-coagulants, opioid receptor agonists and antagonists, and the like, to the extent that it has become a common occurrence for patients suffering from, or susceptible to, such ailments to carry these devices around with them.

With regard to injection pen systems in particular, for example, one of the challenges has been to provide easy to use, reliable and fairly failsafe monitoring and measuring systems that can be adapted to the various different variants of such commercially available pen injection systems, of which there are many. The injection pen system, to which the electronic dose measuring device is adapted and configured for removable attachment, is generally equipped with a proximally located dose setting wheel and an injection activator. The dose setting wheel rotates about a central longitudinal axis of the pen injection system to allow a user to set the dose of medicament for injection. During the dose setting, or dose “dialling” step, the dose setting wheel is generally rotatable in both a clockwise, and a counter-clockwise direction, these directions corresponding generally to an increase in the selected dose, and a decrease in the selected dose, to be administered, respectively, or vice-versa, depending on the manufacturer. The injection activator is often represented by a push-button, usually located proximally of the dose setting wheel, and in the majority of injection pens at the proximal extremity of the injection pen system. After a dose has been set, or “dialled”, as the term is commonly known in the art, when a user of the injection system then presses the injection activator in a distal direction, a piston is driven which is connected to a plunger in order to expel drug from a chamber within the injection pen body out through a needle that the user has inserted into an appropriate injection site, for example, the skin, fatty tissue, or muscle, depending on the type of drug to be administered. The dose setting wheel is sometimes, but not necessarily, also coupled to the injection drive mechanism so that it can, depending on the manufacturer and model of injection pen, also rotate as injection of the drug proceeds. The functioning of such injection systems is well known per se in the art.

The monitoring module is intended for mounting onto a pen injection system in which the dose setting wheel can be configured to either rotate during the ejection/injection phase of operation, or, on the contrary, not rotate during the ejection/injection phase of operation of the pen injection system. For example, the Kwikpen® injection pen mentioned above does not have a dose setting wheel that rotates during injection, whereas the dose setting wheel of the Lantus Solostar® and FlexPen® injection pens do rotate during injection.

Injection monitoring and dose measuring is known generally per se when associated with injection pen systems, and enables users of such pen injection systems, and health care professionals involved in the treatment and follow-up of such patients, to monitor more closely their own injection regimes, and in many cases, the doses actually administered, in an attempt to lead to better healthcare outcomes. These developments have been accompanied by the increased associated use of software and portable communications devices such as tablets or smartphones, which have been programmed to receive information from, and interact with, the monitoring and measuring systems in order to provide information to the user or healthcare professional on-the-fly, or at regular intervals via appropriate communications units included in the monitoring systems. The injection monitoring module is adapted and configured to be removably attached to the proximal end of such an injection pen system. The expressions “removably attached”, “removably attachable”, “removably mounted” or “removably mountable” as might be used in the present specification are to be understood as referring to the possibility of attaching, or mounting, and subsequently removing, or dismounting, or unattaching, the injection monitoring module, for example, in the case of transferring the injection monitoring module to another pen injection system, or for example, if the injection monitoring module is damaged during use and requires replacement. Such attachment and subsequent removability can be achieved by providing coupling means on the injection monitoring module which engage in a releasable manner with the proximal end of the pen injection system, for example via frictional or elastic engagement, or via other releasable fastening means, such as clips, straps, screw threads and corresponding tightening rings, and the like, which engage with either the dose setting wheel, or the injection activator, and/or even the body of the injection pen system.

The applicant has previously filed a number of patent applications relating to removably attachable electronic injection monitoring modules for such handheld injection pen systems, which have been published, for example, WO2020/217076, WO2020/217094, and WO2021/260404A1. Accordingly, the removably attachable injection monitoring modules described in these patent applications, can be generally described as being configured and adapted to be removably attached to a proximal end of an injector pen having an injection activation button included at the proximal end of the injector pen to activate injection. More particularly, the injection monitoring module generally comprises the following structure:
a distal cylindrical body having a longitudinal bore and a central longitudinal axis, the distal cylindrical body being adapted and configured for co-axially mounting, and rotationally engaging with, a proximal dose selector wheel located adjacent to, and distally of, the injection activation button of the pen injection system, the distal cylindrical body further comprising at least one magnetic field producing element located at, or adjacent, a proximal end of the distal cylindrical body; and
a proximal electronic injection monitoring body, comprising an electronic injection monitoring system, the injection monitoring body being configured to be mounted within the bore of the distal cylindrical body, and wherein the electronic injection monitoring body is selectively movable axially along a central longitudinal axis, and free to rotate about said central longitudinal axis.

Such a structure therefore combines an outer distally located body, which engages with, and rotates with a dose setting wheel of a pen injection system during dose setting, and/or optionally during dose ejection, depending on the specific functioning of the injection pen, and an inner, and proximal body containing an electronic injection monitoring system, which inner body is generally free to rotate about central the longitudinal axis, but which is selectively movable axially along said central longitudinal axis within the bore of the distal cylindrical body. The system of the electronic injection monitoring module body is configured to register changes in magnetic field as the magnetic field producing element is rotated about the central longitudinal axis during dose setting and/or dose ejection, depending on the functioning of the injection pen, but also uses the selective axial movement along the central longitudinal axis to register a change in magnetic moment along the central longitudinal axis to also aid in the determination of other operational conditions, such as the start of an ejection of ejectable substance, such as a medicament, from the pen injector. In these circumstances, selective axial movement refers to the operation of the injection pen, for example, by indirectly pressing on the activator push button via a corresponding button element provided at a proximal end of the proximal electronic injection monitoring module body, in order to effect ejection of a substance contained within a cartridge or chamber included in the pen injection system, for example, containing a suitable pharmaceutical or other injectable substance. Similarly, once activation of the push button of the pen injector ceases, for example, due to a user releasing digital pressure on the proximal push button of the electronic dose measuring body, the latter is generally selectively moved in a proximal direction, either through recoil in the pen injection system, or by a return spring provided in the electronic dose measuring body, thereby allowing for the detection or registration by the electronic circuitry of the return to a reset position of the dose measuring device.

In all of the above, the notions of proximal and distal refer to relative positions with regard to any of an injection monitoring system, injection monitoring module, and pen injection system in general, wherein “proximal” relates to a point or position or direction that is generally oriented in the direction towards the holder of the injection monitoring system, injection monitoring module, or pen injection system, and “distal” relates to a point or position or direction that is generally oriented in the direction away from the holder of the injection monitoring system, injection monitoring module, or pen injection system, for example towards a target site for injection, whether that be another part of the user's body, or a different person's, or animal's, body, or simply a target site for ejection of the substance contained within the pen injection system.

As has been mentioned above, the applicant has previously filed and published applications for determining when a dose has been set, or when an injection has been initiated, using an electronic injection monitoring module as generally described above, and which integrates an electronic injection monitoring system having at least one rotatable magnet on the one hand, configured to rotate about a central longitudinal axis of the injection monitoring system, and and at least one magnetometer which is axially translatable along said central longitudinal axis, on the other hand. Nonetheless, the determination, and/or monitoring, of an injection endpoint still represents a number of significant challenges, especially with regard to injection pen systems in which the dose setting wheel does not rotate during injection.

One aspect therefore, of the present invention, is a system, and method, for the monitoring and/or determination of an injection endpoint, of the type that is adapted and configured to be integrated into an injection endpoint monitoring module as described above.

Accordingly, another aspect of the invention is an injection endpoint monitoring system adapted and configured to be integrated into an injection monitoring module that is mounted to a proximal end of a pen injection system comprising a proximal activation button, the injection endpoint monitoring system comprising:
a processor, a magnetometer, and an accelerometer, wherein the magnetometer and accelerometer are electrically connected to the processor, and the processor is configured to receive data and/or information from either the magnetometer and/or the accelerometer;
a magnetic field generator configured to provide a magnetic field detected by the magnetometer, wherein the magnetic field generator is configured to be rotated about a first axis of the system;
wherein the magnetometer is located on, and configured to be moved in translation along, said first axis of the system;
wherein the processor is configured to process:
   data and/or information received from the magnetometer due to rotation of the magnetic field generator about said first axis of the system, and to determine from said data and/or information whether a dose setting operation has been effected;
   data and/or information received from the accelerometer due to a translational movement of the accelerometer along the first axis of the system; and
wherein the processor is configured to process the data and/or information received synchronously from both the magnetometer and the accelerometer to determine an endpoint of an injection operation of the pen injection system.

As referred to above, the term data and/or information relates to signals, values, arrays and the like, which are represented in a form or a structure, known to, or understood by, the processor for subsequent processing. The data and/or information representations can therefore be in a form known per se, which are communicated to, and received by, the processor, from the magnetometer and the accelerometer. For example, the data can be in the form of electrical, optoelectronic, or optical signals, with the processor integrating corresponding circuitry and or programming logic and/or instructions, which are configured to process said signals.

Similarly, as will be generally understood, the magnetic field generator produces a three dimensional magnetic field, and the corresponding magnetic fields produced by the magnetic field generator are normally read or detected in a known manner by the magnetometer, along an x, y and z axis, each of said x, y or z axes being perpendicular one to the other. The data and/or information from the magnetometer readings are communicated to the processor as indicated above.

Magnetic field generators are known per se, for example, classical magnets, electromagnets, and mixed material magnets. Such magnets are typically made from magnetizable materials, having magnetic or paramagnetic properties, whether naturally or when an electric or other energizing flow traverses or affects said material to produce or induce a magnetic field in said material. Suitable materials can be appropriately selected from:
ferrite magnets, especially sintered ferrite magnets, for example, comprising a crystalline compound of iron, oxygen and strontium;
composite materials consisting of a thermoplastic matrix and isotropic neodymium-iron-boron powder;
composite materials made up of a thermoplastic matrix and strontium-based hard ferrite powder, whereby the resulting magnets can contain isotropic, i.e. non-oriented, or anisotropic, i.e. oriented ferrite particles;
composite materials made of a thermo-hardening plastic matrix and isotropic neodymium-iron-boron powder;
magnetic elastomers produced with, for example, heavily charged strontium ferrite powders mixed with synthetic rubber or PVC, and subsequently either extruded into the desired shape or calendered into fine sheets;
flexible calendered composites, generally having the appearance of a brown sheet, and more or less flexible depending on its thickness and its composition. These composites are never elastic like rubber, and tend to have a Shore Hardness in the range of about 40 to about 70 Shore D ANSI. Such composites are generally formed from a synthetic elastomer charged with strontium ferrite grains. The resulting magnets can be anisotropic or isotropic, the sheet varieties generally having a magnetic particle alignment due to calendering;
laminated composites, generally comprising a flexible composite as above, co-laminated with a soft iron-pole plate;
neodymium-iron-boron magnets;
steels made of aluminium-nickel-cobalt alloy and magnetized;
alloys of samarium and cobalt.

Of the above list of magnetic field generators suitable for use in the present invention, those selected from the group consisting of neodymium-iron-boron permanent magnets, magnetic elastomers, composite materials made up of a thermoplastic matrix and strontium-based hard ferrite powder, and composite materials made of a thermo-hardening plastic matrix and isotropic neodymium-iron-boron powder, are preferred. Such magnets are known for their ability to be dimensioned at relatively small sizes whilst maintaining relatively high magnetic field strength.

According to another aspect, the first axis is a z-axis. The z-axis is advantageously coincidental, and coextensive, with a central longitudinal axis of the injection monitoring system and/or injection monitoring module.

According to yet another aspect, the processor is configured to process data received by it from the magnetometer with regard to a selective choice of x-axis, y-axis or z-axis, each axis situated orthogonally one with respect to the others, or a combination of one or more of said axes.

According to yet another aspect, the processor is configured to determine that a dose has been set when:
data and/or information is received from the magnetometer which is indicative of an increase in a resultant magnetic field intensity to one greater than a baseline magnetic field intensity of the system at rest, and less than a maximum resultant magnetic field intensity, the increase resulting from rotation of the magnetic field generator about the first axis.

According to another advantageous aspect, the resultant magnetic field intensity is determined from readings taken from the x-axis and the y-axis. As indicated above, the processor can be advantageously configured accordingly to process data selectively from the x-axis and the y-axis of the magnetometer, for example, in order to determine the resultant magnetic field intensity.

The processor integrates, is programmed with, or is configured to execute, programming logic and/or instructions, for example, stored in a memory, to compare a baseline resultant magnetic field intensity of the system at rest, or series of values representative of said baseline resultant magnetic field intensity, with actual readings received from the magnetometer as the injection monitoring system is used, and to calculate whether the resultant magnetic field intensities determined from the read magnetic field intensities, are less than a maximum resultant magnetic field intensity.

The baseline resultant magnetic field intensity can be either determined empirically, e.g. by measuring the magnetic field intensities generated by any provided magnetic field generator in situ in a monitoring module when mounted on an injection system, or calculated in advance using a mathematical model, from the dimensions and nominal strength of the magnetic field generator, and the at rest position of said magnetic field generator relative to the magnetometers, also taking into account any background magnetic fields, such as the earth's magnetic field (EMF), or any required corrections for errors or offsets.

Similarly, the maximum resultant magnetic field intensity can be either determined empirically, e.g. by measuring the magnetic field intensities generated by any provided magnetic field generator in situ in a monitoring module when mounted on an injection system which is then operated to simulate, or reproduce the beginning of an injection, or calculated in advance using a mathematical model, from the dimensions and nominal strength of the magnetic field generator, and the position of the latter as the magnetometer is moved along said z-axis relative to said magnetic field generator, also taking into account any background magnetic fields, such as the earth's magnetic field (EMF), or any required corrections for errors or offsets. The maximum resultant magnetic field intensity occurs when the magnetometer is moved, or translates, along the z-axis, from an at rest position on the z-axis, to an injection begin position along said z-axis, when injection activation is effected, and as an injection proceeds.

The baseline, or minimum, resultant magnetic field intensity and the maximum resultant magnetic field intensity can be stored in the system, for example, in a memory, for access by the processor as and when required.

The comparison by the processor of the predetermined baseline resultant magnetic field intensity, and the maximum resultant magnetic field intensity, with the actual intensities read from the magnetometer and received by the processor is used to determine whether a dose has been set. This comparison can also optionally be used to determine whether the magnetic field generator has been rotated around the z-axis for some other reason, for example, accidentally.

The dose setting step produces a characteristic sequence of resultant magnetic field intensities, which can be suitably recognized as such by the processor of the system, and for which the processor is correspondingly programmed, or provided with an executable instruction set, for example, stored in a memory, enabling such a sequence to be recognized and considered as the dose setting step.

Insofar as the data and/or information received by the processor from the accelerometer is concerned, this data and/or information can be indicative of a number of situations depending on the measured values, for example, a baseline acceleration of the system, which does not exceed a minimum value—this can be determined empirically or mathematically, for each pen injection system and monitoring module, for example, via hands on testing or modeling, in a manner similar to that for determining the baseline resultant magnetic field intensity and maximum resultant magnetic field intensity. The acceleration value can be suitably stored in a memory which is accessed by the processor as and when required.

The processor suitably integrates, is programmed with, or is configured to execute, programming logic and/or instructions to compare the baseline value of acceleration, or series of values over time, with the actual value or series of values over time, read from the accelerometer, and to make a corresponding determination of an operational status with regard to said comparisons.

Generally, if the acceleration data is not indicative of an increase in acceleration values above the predetermined baseline acceleration value or value range, then the injection monitoring system has not been moved in either a proximal or distal direction along the central longitudinal axis, which is coincidental with the z-axis, and the processor will register such a state in the injection monitoring system. Alternatively, if the processor determines from the comparisons that the acceleration value or range of acceleration values does not exceed the predetermined baseline acceleration value, whilst at the same time an increase in magnetic field reading corresponding to a dose setting operation has been determined by the processor to have occurred, then the processor is configured to register that a dose setting operation has occurred.

When the injection monitoring system is integrated into a corresponding injection monitoring module, the magnetic field generator can be suitably provided by a pair of single dipole magnets, located in corresponding recesses provided in the outer cylindrical body of the injection monitoring module. The magnets can appropriately be rod-shaped or cylindrical dipole magnets, one positioned in opposite polar facing orientation with regard to the other, for example N-S aligning with S-N, with the magnets being positioned to lay flat along their own longitudinal axes, across a horizontal plane that bisects, and is orthogonal to, the central longitudinal axis, each magnet being located on an opposing side of said central longitudinal axis, for example, at 180° of rotation around said central longitudinal axis, one with respect to the other.

Similarly, the electronic circuitry is suitably located within the electronic injection monitoring module body which is positioned in, and translatable along, the central longitudinal axis, and is located, at least in part, proximally of the outer cylindrical body. The electronic circuitry comprises the processor, magnetometer and accelerometer, for example, on an appropriately configured printed circuit board, along with an autonomous, or semi-autonomous power supply, such as lithium ion battery or a rechargeable battery. The processor can, for example, be integrated into a micro-controller which integrates a system clock, that can, for example, be configured to function based on the operational frequency of the processor and/or micro-controller.

According to yet another aspect, the processor is configured to determine that an injection endpoint has been reached when:
data and/or information is received from the magnetometer which is indicative of the maximum resultant magnetic field intensity; and
data and/or information is received from the accelerometer which is indicative of an absence of acceleration along the z-axis for a predetermined duration.

In regard to the above, the term “absence of acceleration” signifies that the accelerometer does not detect, or the processor is configured to ignore, any acceleration value below a predetermined threshold value, which value can be different to the minimum acceleration value, and can be preprogrammed into the system and determined empirically or via mathematical modeling beforehand, for example. Similarly, the expression “predetermined duration” used herein refers to a duration of time generally comprised between about 0.1 second and about 2 seconds.

According to another aspect, the processor is configured to sample the data and/or information received from the magnetometer and/or accelerometer, in a known manner common to signal processing technology, at a frequency comprised from between about 20 Hz to about 400 Hz. According to yet another aspect, the processor is configured to analyze a magnetogram profile of data and/or information received from the magnetometer simultaneously and synchronously with an acceleration profile of data and/or information received from the accelerometer, to determine any predefined operational status of an injection process, for example, a reset position in which the injection monitoring system has returned to an initial pre-dose setting, and/or pre-injection position, in which the magnetometer and accelerometer have been moved in a proximal direction to the pre-dose setting and/or pre-injection position. Alternatively, the processor can be configured to determine, for example, whether or not a partial injection has been effected, for example, in the case where an injection operation was started, and the chamber or cartridge containing the substance to be injected was spent or used up during injection, and wherein therefore only a part of a required and dialed dose of injectable substance could be injected. In such a situation, the processor is configured to store the intermediately received values provided by the magnetometer and accelerometer in order to calculate that only a part of the dialed dose has been administered. This partially administered dose value can then be uploaded over a wireless connection integrated into the monitoring system to, for example, a smartphone application which is configured to provide visual feedback about administration and treatment observance data to the user of the monitoring system. Additionally, the storing of the partial injection data can, once the injection monitoring module has been removed from the spent pen injection system and installed on a new pen injection system with a freshly filled injectable substance chamber or cartridge, the smartphone application can inform the user of the quantity of remaining dose to be dialed on the new pen injection system in order to complete injection of the required total dose.

According to yet another aspect, an injection monitoring module is provided, comprising an injection monitoring system as substantially described herein, wherein:
the magnetic field generator is located on a first cylindrical body having a central longitudinal bore and a central longitudinal axis, the first cylindrical body being configured to rotate about the central longitudinal axis, and
the processor, magnetometer, and accelerometer are located on a second body, the second body being configured to translate within the central longitudinal bore of the first cylindrical body, from a first proximal injection position to a second distal injection position.

According to yet a further aspect, a method for determining an injection endpoint is provided, comprising:
mounting an injection monitoring module, comprising an injection endpoint monitoring system as described, to the proximal end of an injection pen system comprising a proximal activation button;
dialing a dose on the injection pen system;
activating the proximal activation button of the injection pen system;
configuring the processor of the injection endpoint monitoring system to process the data and/or information received synchronously from both a magnetometer and an accelerometer of the injection endpoint monitoring system to determine an endpoint of an injection operation of the pen injection system.

Other aspects of the invention will become evident from the present, or described herein, with regard to the attached figures, provided as an illustrative example, and in which:

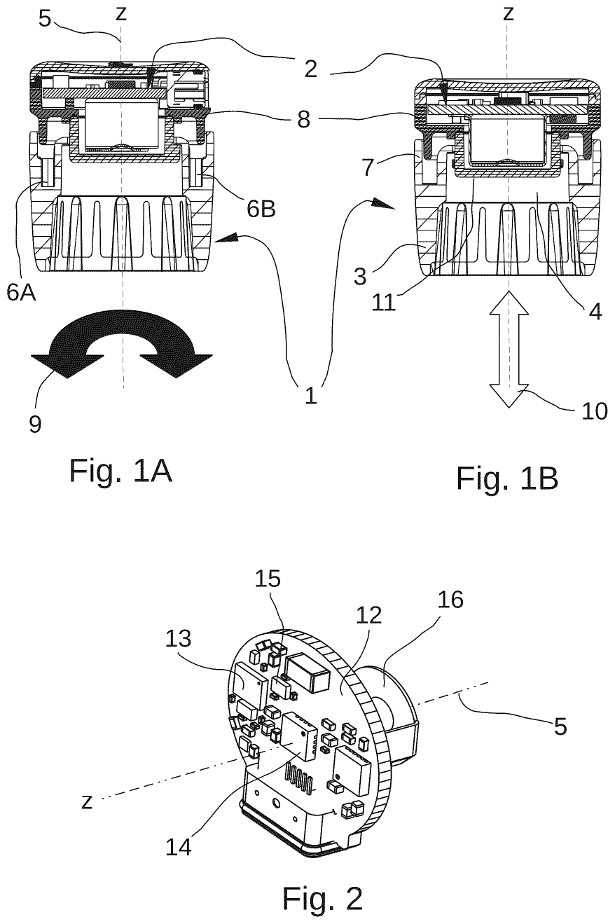

FIGS. 1A and 1B are schematic cross-sectional representations of an injection monitoring module integrating the endpoint injection monitoring system according to the invention;

FIG. 2 is a schematic perspective representation of a detail of a part of an electronic injection monitoring system according to the present invention;

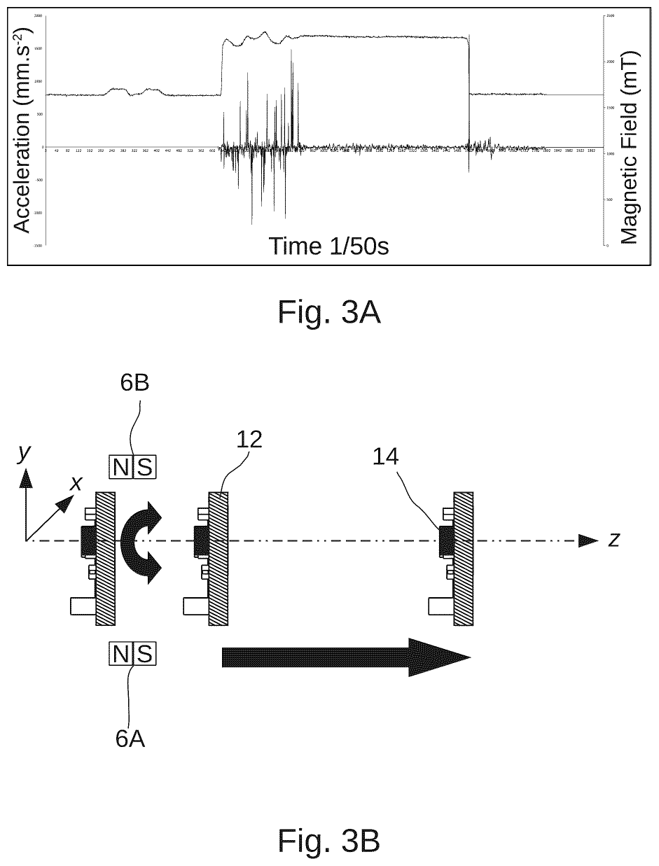

FIG. 3A is a schematic representation of a plot profile of an injection illustrating values of magnetic field and acceleration over time;

FIG. 3B is a schematic representation of the staged movement of the injection monitoring system in synchronization with the profile of FIG. 3A.

### DETAILED DESCRIPTION

Turning now to FIGS. 1A and 1B, an injection monitoring module (1) comprising an injection monitoring system (2) according to the invention has a structure similar to that described in the applicant's previously published PCT applications published as WO2020217076A1, or WO2020217094A1. Briefly stated, the injection monitoring module (1) is configured and adapted to be removably attached to a proximal end of an injector pen (not shown) having an injection activation button included at the proximal end of the injector pen to activate injection. The injection monitoring module (1) generally comprises a distal cylindrical body (3) having a longitudinal bore (4) and a central longitudinal axis (5) which, for the purposes of the present invention is also the a z-axis of the injection monitoring system, as illustrated in FIG. 2 and FIG. 3B. The distal cylindrical body (3) is adapted and configured for co-axially mounting, and rotationally engaging with, a proximal dose selector wheel located adjacent to, and distally of, the injection activation button of the pen injection system (not shown), and also comprises at least one magnetic field generator (6A, 6B) located at, or adjacent, a proximal end (7) of the distal cylindrical body (3). The injection monitoring module (1) also comprises a proximal electronic injection monitoring body (8), comprising the electronic injection monitoring system (2), the electronic injection monitoring body (8) being configured to be mounted within the bore (4) of the distal cylindrical body (3). The electronic injection monitoring body (8) is selectively movable axially along the central longitudinal axis (5, z), and is free to rotate about the central longitudinal axis (5, z). FIGS. 1A and 1B illustrate more specifically the relative positions of the proximal injection monitoring body (8) and the distal cylindrical body (3) with respect to each other during operation of the injection monitoring module. FIG. 1A illustrates the positions when setting or dialing a dose, in which the distal cylindrical body (3) is caused to rotate about the central longitudinal axis (5, z), as indicated by the corresponding arrow (9), and the injection monitoring body (8) is located at maximum proximal distance from the distal cylindrical body (3). FIG. 1B illustrates the relative positions of the distal cylindrical body (3) and injection monitoring body (8) during an injection step (FIG. 1B), in which the proximal injection monitoring body (8) has translated in a distal direction, as per the corresponding arrow (10), to effect an injection by activation of a pen injection system activation button via surface engaging contact between a distal facing surface (11) of the injection monitoring body (8) and the pen injection system activation button. Similarly, the injection monitoring body (8) is configured to translate in a proximal direction once injection has completed, and engaging surface contact between the distal facing surface (11) and the injection button is removed.

FIG. 2 shows some of the detail of an electronic injection monitoring system (2), including a printed circuit board (12), comprising a processor, or micro-controller (13) comprising a processor, a magnetometer (14), an accelerometer (15), and an autonomous power supply holder (16) configured to hold and retain an autonomous power supply, such as lithium ion battery. FIG. 2 also illustrates the virtual superposition of the central longitudinal axis (5, z), which passes through the centre of the magnetometer (14). The micro-controller (13) comprising the processor of the electronic injection monitoring system (2) is configured to receive data and/or information from the magnetometer (14), for example, magnetic field intensities, and/or magnetic field vectors, as the magnetic field generator (6A, 6B), for example a pair dipole magnets, is rotated about the central longitudinal axis (5, z) during dose setting. The magnetic field intensity and/or vector also change as the electronic injection monitoring system (2) translates along the central longitudinal axis (5, z), because the magnetometer (14) moves in a distal direction through the magnetic field produced by the magnetic field generator (5A, 5B) during injection, and moves back through the magnetic field after injection is completed as the injection monitoring system body (8) translates in a proximal direction back to an initial rest, or zero position. The accelerometer (15) captures the acceleration of the electronic injection monitoring body (8), and the processor of the micro-controller (13) is configured to receive the data and/or information structures from the accelerometer (15) as the injection monitoring system body (8) is moved, or translates, along the central longitudinal axis (5, z).

FIG. 3A shows a profile trace over time, or magnetogram, illustrated by the upper plot, of resultant magnetometer intensity along the central longitudinal (5, z) axis for various stages, and statuses of an operation of the injection monitoring system and injection monitoring module (1) integrating such an injection monitoring system (2), and below the magnetogram, an accelerometer profile trace, or plot, or accelerometer readings over the same time span. FIG. 3B shows the corresponding schematic positions and/or movements of the injection monitoring system at each stage of an injection operation. FIG. 3B also shows the corresponding perpendicularly oriented axes x and y with respect to the central longitudinal axis (5, z) and which are associated with the resultant magnetic field intensity generated by the magnetic field generator (6A, 6B).

The initial part of the plot in FIG. 3A shows the system at rest. Both the measured resultant magnetometer (14) intensities and the measured accelerometer (15) values received by the processor are at a respective minimum, i.e. respective baseline value. As can be seen from FIG. 3A, the accelerometer values are substantially equal to 0 m·s⁻², whereas the baseline magnetometer intensities for the exemplified monitoring module and monitoring system are at approximately 1650 micro Teslas. During dose setting, i.e. when the magnetic field generator (5A, 6B), as illustrated by N/S representing the magnetic poles of the magnetic field generators, is rotated about the central longitudinal axis (5, z), as illustrated by the corresponding arrow in FIG. 3B, the magnetometer readings increase above the baseline level, but remain below a maximum. In the example shown in FIG. 3, the dose setting step lasts about 4 to 6 seconds in all, but the time may vary depending on the monitoring module and injection pen system on which the monitoring module is mounted. During this dose setting, or dose dialling, phase, and as can be seen from FIG. 3A, the accelerometer values remain at the accelerometer baseline value.

During an injection step, as shown in FIG. 3B, the injection monitoring system is moved in a distal direction along the central longitudinal axis (5, z) as injection is initiated. This is indicated by the corresponding arrow in FIG. 3B, and is represented in the plot of FIG. 3A by a sudden increase in both the resultant magnetic field intensity, which reaches a maximum, and the accelerometer value which can be both positive and negative. These values remain elevated as injection proceeds. However, as can be seen on the plot in FIG. 3A, the resultant magnetic intensities reported by the magnetometer (14), and the acceleration values reported by the accelerometer (15), to the processor of the injection monitoring system, present a different profile as the system reaches the injection endpoint. This is represented by a substantial absence, or lack of acceleration, whereas the magnetometer (14) readings remain substantially at the maximum over the same period of time. Finally, when a user of the injection system releases the activation button 5 of the injection system, the injection monitoring system is moved, or translates, in a proximal direction, i.e. from right to left on FIG. 3B, along the z-axis. This causes the magnetometer readings to drop to the magnetometer baseline once again, whereas the accelerometer readings indicate a temporary spike.

1. Injection endpoint monitoring system adapted and configured to be integrated into an injection monitoring module that is mounted to a proximal end of a pen injection system comprising a proximal activation button, the injection endpoint monitoring system comprising:
a processor, a magnetometer, and an accelerometer, wherein the magnetometer and accelerometer are electrically connected to the processor, and the processor is configured to receive data and/or information from either the magnetometer and/or the accelerometer;
a magnetic field generator configured to provide a magnetic field detected by the magnetometer, wherein the magnetic field generator is configured to be rotated about a first axis of the system;
wherein the magnetometer is located on, and configured to be moved in translation along, said first axis of the system;
wherein the processor is configured to process:
data and/or information received from the magnetometer due to rotation of the magnetic field generator about said first axis of the system, and to determine from said data and/or information whether a dose setting operation has been effected;
data and/or information received from the accelerometer due to a translational movement of the accelerometer along the first axis of the system; and
wherein the processor is configured to process the data and/or information received synchronously from both the magnetometer and the accelerometer to determine an endpoint of an injection operation of the pen injection system.

2. Injection endpoint monitoring system according to claim 1, wherein the first axis is a z-axis.

3. Injection endpoint monitoring system according to claim 1, wherein the processor is configured to process data received by it from the magnetometer with regard to a selective choice of x-axis, y-axis or z-axis, situated orthogonally one with respect to the others, or a combination of one or more of said orthogonally situated axes.

4. Injection endpoint monitoring system according to claim 1, wherein the processor is configured to determine that a dose has been set when:
data and/or information is received from the magnetometer which is indicative of an increase in a resultant magnetic field intensity greater than a resultant baseline magnetic field intensity of the system at rest, and less than a maximum resultant magnetic field intensity, the increase resulting from rotation of the magnetic field generator about the first axis.

5. Injection endpoint monitoring system according to claim 4, wherein the resultant magnetic field intensity is determined from readings taken from the x-axis and the y-axis.

6. Injection endpoint monitoring system according to claim 1, wherein the processor is configured to determine that an injection endpoint has been reached when:
data and/or information is received from the magnetometer which is indicative of the maximum resultant magnetic field intensity; and
data and/or information is received from the accelerometer which is indicative of an absence of acceleration along the z-axis for a predetermined duration.

7. Injection endpoint monitoring system according to claim 1, wherein the processor is configured to sample the data and/or information received from the magnetometer and/or accelerometer at a frequency comprised from between about 20 Hz to about 400 Hz.

8. Injection endpoint monitoring system according to claim 1, wherein the processor is configured to analyze a magnetogram of data and/or information received from the magnetometer simultaneously and synchronously with an acceleration profile of data and/or information received from the accelerometer, to determine any predefined operational status of an injection process.

9. Injection monitoring module, comprising a system according to claim 1.

10. Injection monitoring module according to claim 11, wherein:
the magnetic field generator is located on a first cylindrical body having a central longitudinal bore and a central longitudinal axis, the first cylindrical body being configured to rotate about the central longitudinal axis, and
the processor, magnetometer, and accelerometer are located on a second body, the second body being configured to translate within the central longitudinal bore of the first cylindrical body, from a first proximal injection position to a second distal injection position.

11. Method for determining an injection endpoint, comprising:
i. mounting an injection monitoring module, comprising an injection endpoint monitoring system according to claim 1, to the proximal end of an injection pen system comprising a proximal activation button;
ii. dialing a dose on the injection pen system;
iii. activating the proximal activation button of the injection pen system;
iv. configuring the processor of the injection endpoint monitoring system to process the data and/or information received synchronously from both a magnetometer and an accelerometer of the injection endpoint monitoring system to determine an endpoint of an injection operation of the pen injection system.

12. Method according to claim 11, comprising configuring the processor of the injection endpoint monitoring system to process the data and/or information received synchronously from both the magnetometer and the accelerometer of the injection endpoint monitoring system, with regard to one or more axes chosen from an x-axis, a y-axis, a z-axis, or a combination of one or more of said axes.

13. Method according to claim 11, wherein the processor is configured to process data and/or information received from the magnetometer and measured along the x-axis and the y-axis, both of which are perpendicular to the z-axis.

14. Method according to claim 11, wherein the processor is configured to determine that a dose has been set when:
data and/or information is received from the magnetometer which is indicative of an increase in resultant magnetic field intensity to one greater than a baseline value and below a predetermined maximum value.

15. Method according to claim 11, wherein the processor is configured to determine that an injection endpoint has been reached when:
data and/or information is received from the magnetometer which is indicative of the maximum resultant magnetic field intensity; and
data and/or information is received from the accelerometer which is indicative of an absence of acceleration along the z-axis for a predetermined duration.

16. Method according to claim 11, wherein the processor is configured to sample the data and/or information received from the magnetometer and/or accelerometer at a frequency comprised from between about 20 Hz to about 400 Hz.

17. Method according to claim 11, wherein the processor is configured to analyze a magnetogram profile of data and/or information received from the magnetometer simultaneously and synchronously with an acceleration profile of data and/or information received from the accelerometer, to determine any predefined operational status of an injection process.
